(19) United States
(12) Patent Application Publication
(10) Pub. No.: US 2024/0279748 A1
(43) Pub. Date: Aug. 22, 2024

(54) EARLY DETECTION AND INTERVENTION OF AT-RISK SUBJECTS FOR PRODROMAL PARKINSON'S DISEASE

(71) Applicant: The Chinese University of Hong Kong, Shatin (CN)

(72) Inventors: Yun Kwok WING, Shatin (CN); Bei HUANG, Ma On Shan (CN); Hei Ming LAI, Ma On Shan (CN); Kay Sheung Paul CHAN, Tai Po (CN); Ka Leung CHAN, Tai Po (CN); Chung Tong Vincent MOK, Hong Kong SAR (CN)

(21) Appl. No.: 18/443,879

(22) Filed: Feb. 16, 2024

Related U.S. Application Data
(60) Provisional application No. 63/446,304, filed on Feb. 16, 2023.

(51) Int. Cl. C12Q 1/689; A61K 45/06; C12Q 1/6869; C12R 1/145; G16H 50/20

(52) U.S. Cl. CPC C12Q 1/689; A61K 45/06; C12Q 1/6869; G16H 50/20; C12Q 2600/118; C12R 2001/145

(57) ABSTRACT

The present invention provides for compositions and methods for the early detection of prodromal Parkinson's Disease as well as for early medical intervention, especially among individual who may not have been diagnosed but are at an elevated risk of later developing Parkinson's Disease and related conditions.

### RELATED APPLICATIONS

This application claims priority to U.S. Provisional Patent Application No. 63/446,304, filed Feb. 16, 2023, the contents of which are hereby incorporated by reference in the entirety for all purposes.

### BACKGROUND OF THE INVENTION

With aging population, neurodegenerative disorders such as Parkinson's Disease (PD) are becoming more prevalent worldwide and present a serious public health concern.

For example, in 2015, over 6 million people were affected by Parkinson's disease, resulting in nearly 120,000 deaths globally. These numbers are expected to double by the year 2030. Typically occurring in older individuals, Parkinson's Disease afflicts about one percent of those over the age of 60. Men are more often affected than women at a ratio of about 3:2 and some early onset of Parkinson's Disease can be seen among individuals before the age of 50. At present, there is no cure for these progressive neurodegenerative disorders, the effects of which are profound and devastating on those afflicted, their family, and the society as a whole. In the case of Parkinson's Disease, all currently available treatment remedies merely aim at alleviating symptoms of the disease. One's average life expectancy following the diagnosis of Parkinson's Disease is between 7 and 15 years.

Accordingly, there exists an urgent need for new and meaningful methods to facilitate the early detection of these neurodegenerative diseases, preferably prior to the emergence of classical symptoms based on which these diseases are diagnosed following the current diagnostic criteria, in hope for achieving effective therapeutic benefits by way of early medical intervention. The purpose of this study is to identify gut microbial species that are relevant to the onset of neurodegenerative diseases, especially at a pre-symptomatic stage, and therefore can provide diagnostic value for these diseases. The present invention fulfills this and other related needs.

### BRIEF SUMMARY OF THE INVENTION

The present inventors discovered in their studies the certain gut microbial species, especially certain bacterial species, are directly relevant to the onset and/or progression of a disease within the category of α-synucleinopathy, e.g., Parkinson's Disease, thus provide an important utility in the early detection of such diseases. The gut microorganisms so identified now serve to provide new methods and compositions useful for the diagnosis of progressive neurodegenerative diseases at a critical early stage such as prodromal Parkinson's Disease.

In a first aspect, the present invention provides a method for diagnosing prodromal Parkinson's Disease in a subject based on demographic features (age and sex) and the presence and quantity of eight bacterial species Roseburia faecis, Faecalibacterium prausnitzii, Clostridium_fessum, Dysosmobacter_welbionis, Lachnospira_pectinoschiza, Clostridium_sp_AM22_11AC, Anaerotruncus_colihominis, and Clostridiales_Family_XIII_bacterium_BX16. The method include these steps: first, determining in a fecal sample taken from the subject the amount of each of the bacterial species according to the following designations: Roseburia faecis (Species01), Faecalibacterium prausnitzii (Species02), Clostridium_fessum (Species03), Dysosmobacter_welbionis (Species04), Lachnospira_pectinoschiza (Species05), Clostridium_sp_AM22_11AC (Species06), Anaerotruncus_colihominis (Species07), and Clostridiales_Family_XIII_bacterium_BX16 (Species08); second, determining the age (years) and biological sex (male or female) of the subject at the time of fecal sample collection; then calculating a probability score:

$$Sex = 1 if male = 2 i f female$$

$$X = 0.773 - 0.181 * Species 01 - 0.103 * Species 02 - 0.638 * Species 03 + 2.334 * Species 04 - 0.275 * Species 05 - 0.543 * Species 06 + 10.336 * Species 07 + 2.005 * Species 08 - 0.006 * age + 0.565 * sex$$

$$Probability = [ 1 / ( 1 - {e}^{- X} ) ] * 100$$

and lastly determining presence of prodromal Parkinson's Disease or an elevated risk for prodromal Parkinson's Disease in the subject when the probability score from the above calculation is at least 50%, or determining absence of prodromal Parkinson's Disease or no elevated risk for prodromal Parkinson's Disease in the subject when the probability score is less than 50%. In some embodiments, the first step comprises DNA extraction and metagenomic sequencing. In some embodiments, the amount of each of the eight bacterial species is the relative abundance of each of the bacteria. In some embodiments, the subject being tested has a family history of Parkinson's Disease (especially prodromal Parkinson's Disease) but has not received a diagnosis of Parkinson's Disease. In some embodiments, the subject being tested is at least 40, 50, or 60 years of age. Optionally, the method further includes a treatment step upon determining presence of prodromal Parkinson's Disease or an elevated risk for prodromal Parkinson's Disease in the subject. For example, the treating step may include administering to the subject one or more compositions comprising an effective amount of one or more therapeutic agents approved for use to treat Parkinson's Disease (e.g., one or more dopamine agonists, dopamine metabolism blockers, levodopa metabolism blockers, and adenosine blockers) and prodromal Parkinson's disease (e.g., clonazepam and melatonin) according to methods as described herein or known in the pertinent medical field.

In a second aspect, the present invention provides a kit for detecting prodromal Parkinson's Disease in a subject comprising one or more reagents for determining the amount of each of bacterial species Roseburia faecis, Faecalibacterium prausnitzii, Clostridium_fessum, Dysosmobacter_welbionis, Lachnospira_pectinoschiza, Clostridium_sp_AM22_11AC, Anaerotruncus_colihominis, and Clostridiales_Family_XIII_bacterium_BX16. In some embodiments, the reagents comprise those for extracting a sufficient amount of good quality DNA from the fecal sample. In some embodiments, the reagents include those suitable for use in performing metagenomic sequencing.

### BRIEF DESCRIPTION OF THE DRAWINGS

FIG. 1. Summary of different stages of Parkinson's Disease (PD) and current evidence of gut dysbiosis at prodromal PD.

FIG. 2. Flow chart of early detection of high-risk population of alpha-synucleinopathies based on gut microbial features.

FIG. 3. The performance of present invention in diagnosing prodromal Parkinson's disease in discovery and validation cohorts.

### DEFINITIONS

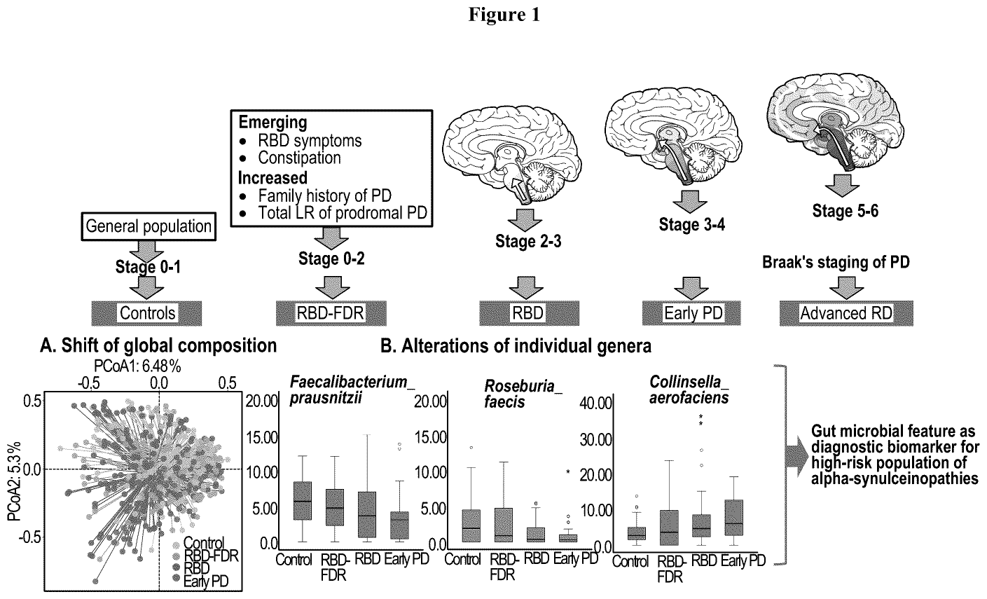

As used herein, the term “Parkinson's Disease” or “PD” refers to a progressive degenerative disorder of the central nervous system that primarily affects a patient's motor functions, presented as tremor, rigidity, slowness of movement, and difficulty with walking. The symptoms usually emerge gradually, and some non-motor symptoms occur much earlier than motor dysfunctions. Cognitive and behavioral problems may exhibit as depression, anxiety, and apathy, and dementia in patients during the course of the illness, albeit the clinical diagnosis of Parkinson's disease (PD) focuses on motor impairment. Currently accepted diagnostic criteria require the presence of bradykinesia and at least 1 other motor sign, such as rigidity or rest tremor, which is commonly referred to as motor-PD. On the other hand, neurodegeneration begins decades before the appearance of motor signs, often with the development of nonmotor symptoms such as hyposmia and sleep disturbance. The clinical staging model of PD as proposed by Dr. Braak (FIG. 1) posits the spread of Lewy pathology in a caudal to rostral pattern. Accordingly, stages 1 and 2 represent early pathology in the olfactory bulb and brainstem, producing prodromal symptoms. Stage 3 represents involvement of the substantia nigra to produce motor symptoms. Stages 4 to 6 indicate increasing degrees of cortical involvement, associated with increasing rates of psychosis and dementia. The long latent phase of Parkinson's Disease during stages 1-2 is termed “prodromal Parkinson's Disease” (or “prodromal-PD”).

The term “α-synucleinopathy” is used to refer to a collection of neurodegenerative diseases characterized by the abnormal accumulation of insoluble fibrillary α-synuclein protein aggregates in the central and/or peripheral nervous system such as in the neurons, nerve fiber, or glial cells. Aside from three main types of α-synucleinopathy, Parkinson's disease (PD), dementia with Lewy bodies (DLB), and multiple system atrophy (MSA), this term also encompasses incidental Lewy body disease.

The term “inhibiting” or “inhibition,” as used herein, refers to any detectable negative effect on a target biological process, such as RNA/protein expression of a target gene, the biological activity of a target protein, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process (e.g., a subject's bodyweight, or the blood glucose/cholesterol level, or any other measurable symptom or biomarker in a subject, such as the level or relative amount of a microorganism in a subject's gastrointestinal tract), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or parameter.

As used herein, the term “treatment” or “treating” includes both therapeutic and

preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

The term “severity” of a disease refers to the level and extent to which a disease progresses to cause detrimental effects on the well-being and health of a patient suffering from the disease, such as short-term and long-term physical, mental, and psychological disability, up to and including death of the patient. Severity of a disease can be reflected in the nature and quantity of the necessary therapeutic and maintenance measures, the time duration required for patient recovery, the extent of possible recovery, the percentage of patient full recovery, the percentage of patients in need of long-term care, and mortality rate.

A “patient” or “subject” receiving the composition or treatment method of this invention is a human, including both adult and juvenile human, of any age, gender, and ethnic background, who may not have been diagnosed with any particular disease or disorder (e.g., have not had been given a diagnosis of any α-synucleinopathy such as Parkinson's Disease) but are in need of reducing disease risk due to known risk factors (e.g., to reduce or eliminate risk for developing Parkinson's Disease in someone with constipation). Typically, the patient or subject receiving treatment according to the method of this invention is not otherwise in need of treatment by the same therapeutic agents. For example, if a subject is receiving the symbiotic composition according to the claimed method, the subject is not suffering from any disease that is known to be treated by the same therapeutic agents. Although a patient may be of any age, in some cases the patient is at least 40, 45, 50, 55, 60, 65, 70, 75, 80, or 85 years of age; in some cases, a patient may be between 40 and 49 years old, or between 50 and 60 years of age, or between 60 and 85 years of age. A “child” subject is one under the age of 18 years, e.g., about 5-17, 9 or 10-17, or 12-17 years old, whereas an “adult” subject is one who is 18 years or older.

The term “effective amount,” as used herein, refers to an amount that produces intended (e.g., therapeutic or prophylactic) effects for which a substance is administered. The effects include the prevention, correction, or inhibition of progression of the symptoms of a particular disease/condition and related complications to any detectable extent, e.g., incidence of disease, time of onset, severity and number of the symptoms of the disease and related disorders (e.g., Parkinson's Disease). The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art. Science and Technology of Pharmaceutical Compounding (1999); and Pickar, Dosage Calculations (1999)).

The term “about” when used in reference to a given value denotes a range encompassing ±10% of the value.

### DETAILED DESCRIPTION OF THE INVENTION

### I. Introduction

There is increasing recognition that neurodegeneration begins decades before the appearance of motor signs, often with the development of nonmotor symptoms such as hyposmia and sleep disturbances. Braak staging of Parkinson's Disease (FIG. 1) posits the spread of Lewy pathology in a caudal to rostral pattern. In this model, stages 1 and 2 represent early pathology in the olfactory bulb and brainstem, producing prodromal symptoms. Stage 3 represents involvement of the substantia nigra to produce motor symptoms. Stages 4 to 6 indicate increasing degrees of cortical involvement, associated with increasing rates of psychosis and dementia. The long latent phase of Parkinson's Disease (PD), termed prodromal-PD, represents an opportunity for early recognition of incipient PD. Such early recognition can allow not only better prognostic counseling but also initiation of possible neuroprotective therapies at a stage when therapies might be most effective. This invention describes the process by which gut microbial species are identified as relevant to neurodegenerative disorder, especially prodromal PD, enabling early detection of presence of the condition in an individual or risk of developing the condition at a later time, thus enabling early intervention for optimal outcome.

The invention includes these steps: fecal sample collection, nucleic acid extraction, and quantification of gut microbes. All of these steps are non-invasive and without contraindications or side effects. The preferred detection/quantitation technology, metagenomic sequencing, is very mature and can be readily carried out in research institutions around the world.

### II. Detection of Gut Bacterial Species

The present inventors discovered that the presence and quantity of certain bacterial species in an individual's gastrointestinal tract can be indicative of the presence of an α-synucleinopathy (such as Parkinson's Disease and prodromal Parkinson's Disease) or the risk of developing the disease at a later time. Thus, the first aspect of the present invention relates to the detection, measurement, and analysis of these gut bacteria species in the context of disease diagnosis or prognosis.

The presence and quantity of gut bacterial species are readily determined by analyzing a test individual's stool samples. Upon collection of stool samples, nucleic acids, especially DNA, are first extracted. Quantitative analysis can then be carried out to measure the level of total nucleic acids (e.g., DNA) derived from all bacteria species present in a sample and/or the relative abundance of certain bacteria species present in the sample.

Various methods have been reported in the literature for determining the levels of all bacterial species in a sample, for example, amplification (e.g., by polymerase chain reaction or PCR) and sequencing of bacterial polynucleotide sequence taking advantage of the sequence similarity in the commonly shared 16S rRNA bacterial sequences or shotgun metagenomics that sequencing of all genomics present in a sample. For the metagenomics, the level of any given bacterial species may be determined by dividing the number of reads aligned to a taxonomy by the total number of reads of a sample. A percentage abundance is sometimes used as a parameter to indicate the relative level of a bacterial species in a given environment (e.g., in an individual's gut as represented by a stool sample).

### III. Analysis of Gut Bacterial Species

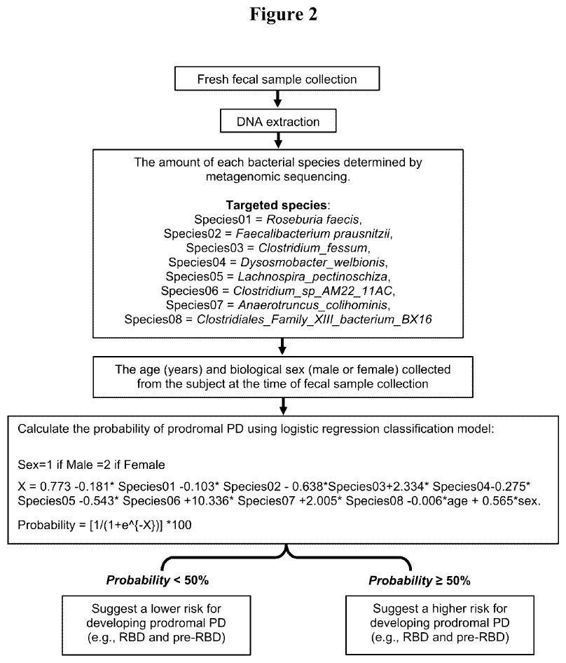

The present inventors discovered that the presence and quantity of certain bacteria species in an individual's gastrointestinal tract, as represented in a stool sample taken from the individual, can provide information indicative of the presence of an α-synucleinopathy (such as Parkinson's Disease, especially prodromal Parkinson's Disease) in the individual. More specifically, the presence and quantity of eight bacteria species Roseburia faecis, Faecalibacterium prausnitzii, Clostridium_fessum, Dysosmobacter_welbionis, Lachnospira_pectinoschiza, Clostridium_sp_AM22_11_AC, Anaerotruncus_colihominis, and Clostridiales_Family_XIII_bacterium_BX16 in a test subject stool sample are first determined, and the level of each bacteria species is analyzed using a logistic regression classification model according to the flow chart shown in FIG. 2 in order to generate a probability score. When the score is at least 50%, the subject is deemed to have prodromal Parkinson's Disease or have an elevated risk for later developing Parkinson's Disease. Conversely, when the score is less than 50%, the subject is deemed to not have prodromal Parkinson's Disease and not have any elevated risk for later developing Parkinson's Disease.

More specifically, the eight bacterial markers are designated as follows: Roseburia faecis (Species01), Faecalibacterium prausnitzii (Species02), Clostridium_fessum (Species03), Dysosmobacter_welbionis (Species04), Lachnospira_pectinoschiza (Species05), Clostridium_sp_AM22_11AC (Species06), Anaerotruncus_colihominis (Species07), and Clostridiales_Family_XIII_bacterium_BX16 (Species08). The relative abundance of each of these eight bacteria markers is determined by quantitative methods such as metagenomic sequencing.

The probability score is then calculated based on the relative abundance of the bacterial markers and the demographic characteristics of the subjects (age and biological sex) according to the equation shown below:

$$Sex = 1 if Male ; = 2 i f female$$

$$X = 0.773 - 0.181 * Species 01 - 0.103 * Species 02 - 0.638 * Species 03 + 2.334 * Species 04 - 0.275 * Species 05 - 0.543 * Species 06 + 10.336 * Species 07 + 2.005 * Species 08 - 0.006 * age + 0.565 * sex$$

$$Probability = [ 1 / ( 1 - e \hat{} \{ - X \} ) ] * 100$$

If the calculated probability is ≥50%, the test subject is classified as having prodromal PD or at high risk for prodromal PD. Otherwise (probability <50%), the subject is classified as non-prodromal PD case and no increased risk for prodromal PD.

### IV. Treatment for Parkinson's Disease

Known therapeutic agent or agents may be used in the practice of the present invention for the purpose of treating prodromal Parkinson's Disease or for the prevention/risk reduction of the onset of Parkinson's Disease among individuals who have been identified as having prodromal PD or at high risk for prodromal PD following the methods of this invention described above and herein. In such applications, one or more of therapeutic agents previously already in use for Parkinson's Disease, including prodromal Parkinson's Disease, can be administered to patients concurrently with an effective amount of the active agent(s) either together in a single composition or separately in two or more different compositions.

For example, drugs and supplements that are known to have been used to treat Parkinson's Disease include various dopamine agonists, dopamine metabolism blockers, levodopa metabolism blockers, and adenosine blockers. They may be used in accordance with the present invention to treat Parkinson's Disease so as to reduce an individual's risk of acute onset of symptoms, delay the onset, alleviate pertinent symptoms, and/or reduce potential disease severity (including morbidity and mortality). In particular, known drug/supplement or nutritheutical combinations including one or more dopamine analogs/stimulators, enzyme inhibitors suppressing dopamine metabolism (e.g., MAO-B inhibitors and COMT inhibitors), amantadine, and anticholinergic drugs may be useful for the practice of the present invention either in one combined composition or in several compositions, to be administered in a general time frame, e.g., within about 6, 12, 24, 48, or up to 72 hours.

### V. Kits

In addition, the present invention provides a kit for detecting prodromal Parkinson's Disease in a subject. The kit includes containers containing one or more reagents for determining the amount of each one of the eight bacterial species: Roseburia faecis, Faecalibacterium prausnitzii, Clostridium_fessum, Dysosmobacter_welbionis, Lachnospira_pectinoschiza, Clostridium_sp_AM22_11AC, Anaerotruncus_colihominis, and Clostridiales_Family_XIII_bacterium_BX16. For example, the kit may contain one or more reagents for extracting a sufficient quantity of good quality DNA from a stool sample. In the alternative, the kit may contain one or more reagents suitable for use in metagenomic sequencing that allows the user to determine the presence and quantity of each of the above-identified bacterial species.

### EXAMPLES

The following examples are provided by way of illustration only and not by way of limitation. Those of skill in the art will readily recognize a variety of non-critical parameters that could be changed or modified to yield essentially the same or similar results.

### Introduction

Alpha-synucleinopathies, including Parkinson's disease (PD), is characterized by abnormal aggregation of alpha-synuclein (α-syn) protein in the central nervous system (CNS).¹ As the most common type of alpha-synucleinopathy, PD affects around 1% of population aged 60 or above.² Up to this date, however, there has been no known disease-modifying treatment for PD or other alpha-synucleinopathies. Increasing evidence suggests that patients with REM sleep behavior disorder (RBD) and first-degree relatives of RBD (RBD-FDR) are specific representatives of prodromal PD.³ According to the pathologic staging model of PD as proposed by Braak et al., patients with early PD and RBD are corresponding to Braak stage 3/4 and 2/3, while RBD-FDR, a pre-RBD stage, may refer to a pathologic stage earlier than RBD (i.e., Braak stage 0/1/2).^{4,9} Therefore, early detection of these at-risk subjects and perform effective interventions within this critical prodromal stages of PD are of great significance for preventing and delaying the occurrence of Parkinson's disease and probably other alpha-synucleinopathies (FIG. 1).

Importantly, accumulating evidence suggests that alpha-synucleinopathy may originate in the enteric nervous system (ENS) and spread from ENS to central nucleus through the vagus nervous.⁵ Moreover, over the past decade, significant gut dysbiosis has been found in patients with PD, and the altered composition and functional profile of gut microbiota may play critical roles in the pathogenesis of ENS alpha-synucleinopathy.^{6,7} Specifically, it has been hypothesied that PD-associated gut dysbiosis, such as the depletion of short-chain fatty acid (SCFA)-producing bacteria, enrichment of genera Akkermansia, and increased biosynthesis of lipopolysaccharides (LPS), can lead to intestinal hyperpermeability, activation of enteric and systemic immune responses and subsequent enteric α-syn pathology.⁶⁻⁸

It remains unclear, however, whether the above-mentioned PD-associated gut dysbiosis has occurred at prodromal phase before the full-blown development of clinical PD. In this regard, our group further investigated gut microbial composition in patients with RBD and their relatives using 16S ribosomal RNA sequencing.⁹ Similar to patients with PD, RBD patients showed distinct gut dysbiosis, which were characterized by the alterations of overall gut microbial composition, decreased SCFA-producing bacteria (e.g., Roseburia, Lachnospiraceae_ND3007_group, Lachnospira, [Eubacterium]_ventriosum_group, Butyricicoccus, Faecalibacterium, and Lachnospiraceae), and increased hydrogen sulfide-producing Desulfovibrio, mucin-degrading Akkermansia, Collinsella, Oscillospiraceae_UCG-002 and -005. Interestingly, the enrichment of pro-inflammatory Collinsella and depletion of butyrate-producing [Eubacterium]_ventriosum_group has already emerged in RBD-FDR, an earlier pre-RBD stage of alpha-synucleinopathy, after adjusting all potential confounders.

Further metagenomic analysis in a total of 650 subjects showed that Lachnospiraceae_bacterium_NSJ_29, Dysosmobacter_welbionis, Hungatella_hathewayi, Ruthenibacterium_lactatiformans, GGB9699_SGB15216, Methanobrevibacter_smithii, Sphingomonas_sp_FARSPH enriched in RBD-FDR, RBD or early PD, while species Faecalibacterium_prausnitzii. Roseburia_faecis. Sutterella_wadsworthensis, Clostridiales_Family_XIII_bacterium_BX16, Clostridium_fessum, Christensenella_hongkongensis, and Clostridium_sp_AM22_11AC were more abundant in controls.

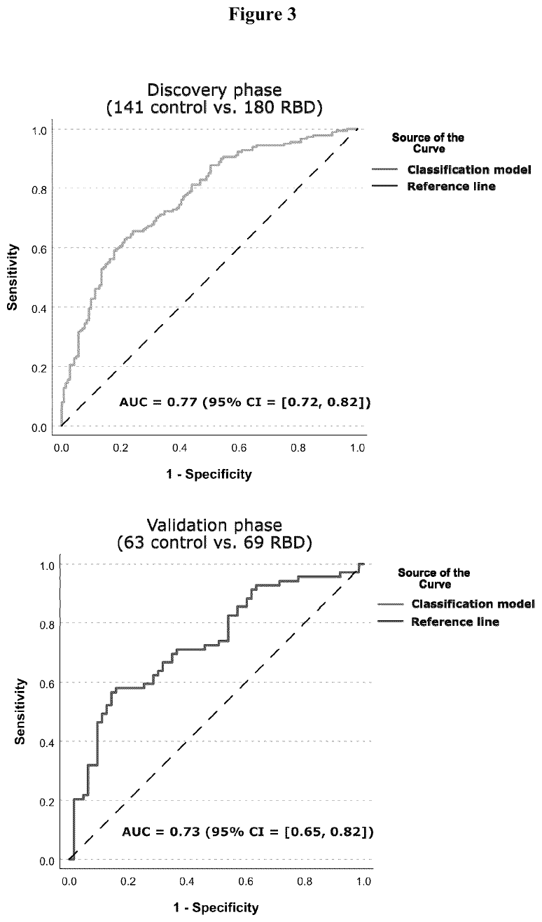

When using a logistic regression classification model, we found that age, sex, and eight gut microbes (Roseburia faecis, Faecalibacterium prausnitzii, Clostridium_fessum, Dysosmobacter_welbionis, Lachnospira_pectinoschiza, Clostridium_sp_AM22_11AC, Anaerotruncus_colihominis, and Clostridiales_Family_XIII_bacterium_BX16) can be used to successfully discriminate RBD from healthy controls with an area under the curve (AUC) of 0.77 (95% CI=[0.72, 0.82]) in the discovery cohort (141 control vs. 180 RBD) and an AUC of 0.73 (95% CI=[0.65, 0.82]) in an independent validation cohort (63 control vs. 69 RBD) (FIG. 3).

In this disclosure, we describe a novel diagnostic biomarker based on the differential features of gut microbiota between prodromal PD and healthy controls, to facilitate early detection of at-risk subjects for prodromal PD from general population. This biomarker comprises relative abundance of eight differential species derived from the metagenomic sequencing and can divide subjects into those with higher and lower risk of prodromal PD by further combining demographic features of the subjects (age and biological sex).

### Materials and Methods

In this invention, we used gut microbial biomarkers identifying at-risk subjects for prodromal PD from general population. Detailed screening steps include sample collection, relative quantification of gut microbes, and risk assessment (FIG. 2).

### 1. Sample Collection

Fresh fecal samples are first collected and stored in sterile containers, and then transferred to the laboratory in a stool kit (containing ice gel, polystyrene box, and thermal bag). Specifically, in this invention, subjects would not be expected to have had (1) antibiotic treatment within one month; (2) have bowel discomfort (for example, diarrhea, vomit, stomachache, etc.) within three days; (3) travel outside of Hong Kong within one week; or (4) have any other situations that might perturb the microbial community. They may be reconsidered after the aforementioned episodes have passed.

### 2. Relative Quantification of Gut Microbes

Quantification of specific gut microbes are analyzed by metagenomic sequencing. DNA extraction is performed using the DNeasy PowerSoil Pro DNA Kit (Qiagen). Taxonomic profiling of gut microbial community is performed using Metagenomic Phylogenetic Analysis (MetaPhlAn 4.0.6). The relative abundance of bacterial species are determined by dividing the number of reads aligned to a taxonomy by the total number of reads of a sample.

### 3. Calculation of Probability of Prodromal PD

The probability of prodromal PD will be calculated based on a pre-trained logistic regression classification model. The detailed equation could be found in FIG. 2. If the calculated probability is ≥50%, indicating a higher risk for developing prodromal PD (e.g., RBD), in other words, these subjects are classified as prodromal PD. Otherwise (probability <50%), it will indicate a relatively low risk to develop prodromal PD (i.e., classified as non-prodromal PD).

### Discussions

This invention provides a new diagnostic biomarker for detecting high-risk population of prodromal PD.

In clinical practice and research, several studies have reported promising biomarkers in detecting RBD patients (i.e., prodromal PD), including alpha-synuclein pathology in tissue biopsy,^{10,11} detection alpha-synuclein from cerebrospinal fluid (CSF)¹² and neuroimaging markers.^{13,14} These biomarkers are capable of distinguishing RBD from controls with a sensitivity ranging from 23.5 to 90.4%, and a specificity ranging from 80 to 100%. There is no biomarker targeting specific gut microbial species for the detection of alpha-synucleinopathies at high-risk populations.

All patents, patent applications, and other publications, including GenBank Accession Numbers and equivalents, cited in this application are incorporated by reference in the entirety for all purposes.

### REFERENCES

1. Spillantini M G, Schmidt M L, Lee V M Y, Trojanowski J Q, Jakes R, Goedert M. α-Synuclein in Lewy bodies. Nature 1997; 388:839-840.
2. Tysnes O-B, Storstein A. Epidemiology of Parkinson's disease. Journal of Neural Transmission 2017; 124:901-905.
3. Liu Y, Zhang J, Lam S P, et al. A case-control-family study of idiopathic rapid eye movement sleep behavior disorder. Ann Neurol 2019; 85:582-592.
4. Braak H, Del Tredici K, Rub U, de Vos R A, Jansen Steur E N, Braak E. Staging of brain pathology related to sporadic Parkinson's disease. Neurobiol Aging 2003; 24:197-211.
5. Kim S, Kwon S H, Kam T I, et al. Transneuronal Propagation of Pathologic alpha-Synuclein from the Gut to the Brain Models Parkinson's Disease. Neuron 2019; 103:627-641 e627.
6. Forsyth C B, Shannon K M, Kordower J H, et al. Increased intestinal permeability correlates with sigmoid mucosa alpha-synuclein staining and endotoxin exposure markers in early Parkinson's disease. PLOS One 2011; 6:e28032.
7. Kelly L P, Carvey P M, Keshavarzian A, et al. Progression of intestinal permeability changes and alpha-synuclein expression in a mouse model of Parkinson's disease. Movement disorders: official journal of the Movement Disorder Society 2014; 29:999-1009.
8. Nishiwaki H, Ito M, Ishida T, et al. Meta-Analysis of Gut Dysbiosis in Parkinson's Disease. Movement disorders: official journal of the Movement Disorder Society 2020.
9. Huang B, Chau S W H, Liu Y, et al. Gut microbiome dysbiosis across early Parkinson's disease, REM sleep behavior disorder and their first-degree relatives. Nat Commun 2023; 14:2501.
10. Sprenger F S, Stefanova N, Gelpi E, et al. Enteric nervous system alpha-synuclein immunoreactivity in idiopathic REM sleep behavior disorder. Neurology 2015; 85:1761-1768.
11. Al-Qassabi A, Tsao T S, Racolta A, et al. Immunohistochemical Detection of Synuclein Pathology in Skin in Idiopathic Rapid Eye Movement Sleep Behavior Disorder and Parkinsonism. Mov Disord 2021; 36:895-904.
12. Iranzo A, Fairfoul G, Ayudhaya A C N, et al. Detection of α-synuclein in CSF by RT-QuIC in patients with isolated rapid-eye-movement sleep behaviour disorder: a longitudinal observational study. The Lancet Neurology 2021; 20:203-212.
13. Ehrminger M, Latimier A, Pyatigorskaya N, et al. The coeruleus/subcoeruleus complex in idiopathic rapid eye movement sleep behaviour disorder. Brain 2016; 139:1180-1188.
14. Pyatigorskaya N, Gaurav R, Arnaldi D, et al. Magnetic Resonance Imaging Biomarkers to Assess Substantia Nigra Damage in Idiopathic Rapid Eye Movement Sleep Behavior Disorder. Sleep 2017; 40.

1. A method for detecting prodromal Parkinson's Disease in a subject, comprising the steps of:
(1) determining in a fecal sample taken from the subject the amount of each of bacterial species Roseburia faecis (Species01), Faecalibacterium prausnitzii (Species02), Clostridium_fessum (Species03), Dysosmobacter_welbionis (Species04), Lachnospira_pectinoschiza (Species05), Clostridium_sp_AM22_11AC (Species06), Anaerotruncus_colihominis (Species07), and Clostridiales_Family_XIII_bacterium_BX16 (Species08);
(2) calculating a probability by the amount of each bacterial species from step (1) and the subject's age (years) and sex:
$$Sex = 1 if male = 2 i f female$$
$$X = 0.773 - 0.181 * Species 01 - 0.103 * Species 02 - 0.638 * Species 03 + 2.334 * Species 04 - 0.275 * Species 05 - 0.543 * Species 06 + 10.336 * Species 07 + 2.005 * Species 08 - 0.006 * age + 0.565 * sex$$
$$Probability = [ 1 / ( 1 - {e}^{- X} ) ] * 100$$
and
(3) determining presence of prodromal Parkinson's Disease or an elevated risk for prodromal Parkinson's Disease in the subject when the probability from step (2) is at least 50%, or determining absence of prodromal Parkinson's Disease or no elevated risk for prodromal Parkinson's Disease in the subject when the probability from step (2) is less than 50%.

2. The method of claim 1, wherein step (1) comprises metagenomic sequencing.

3. The method of claim 1, wherein the amount of each of the eight bacterial species is the relative abundance of each of the bacteria.

4. The method of claim 1, wherein the subject has a family history of Parkinson's Disease but has not received a diagnosis of Parkinson's Disease.

5. The method of claim 1, further comprising treating prodromal Parkinson's Disease upon determining presence of prodromal Parkinson's Disease or an elevated risk for prodromal Parkinson's Disease in the subject.

6. The method of claim 5, wherein the treating step comprises administering to the subject one or more compositions comprising an effective amount of a dopamine agonist, dopamine metabolism blocker, levodopa metabolism blocker, or adenosine blocker.

7. A kit for detecting prodromal Parkinson's Disease in a subject comprising one or more reagents for determining the amount of each of bacterial species of Roseburia faecis, Faecalibacterium prausnitzii, Clostridium_fessum, Dysosmobacter_welbionis, Lachnospira_pectinoschiza, Clostridium_sp_AM22_11AC, Anaerotruncus_colihominis, and Clostridiales_Family_XIII_bacterium_BX16.

8. The kit of claim 7, wherein the one or more reagents are reagents for metagenomic sequencing.
